(19) United States
(12) Patent Application Publication
(10) Pub. No.: US 2023/0009337 A1
(43) Pub. Date: Jan. 12, 2023

(54) Liberty Vitamins and Drinks

(71) Applicant: Stephen Paul Black, Stafford, VA (US)

(72) Inventor: Stephen Paul Black, Stafford, VA (US)

(21) Appl. No.: 17/730,129

(22) Filed: Apr. 26, 2022

Related U.S. Application Data
(60) Provisional application No. 63/259,221, filed on Jun. 30, 2021.
(60) Provisional application No. 63/258,984, filed on Jun. 14, 2021.
(60) Provisional application No. 63/258,907, filed on Jun. 7, 2021.

(51) Int. Cl. A61K 9/00; A61K 31/4745; A61K 31/59; A61K 31/375; A61K 33/30

(52) U.S. Cl. CPC A61K 9/0095; A61K 31/4745; A61K 31/59; A61K 31/375; A61K 33/30

(57) ABSTRACT

I am a practicing physician who has not only recommended these same vitamin ingredients to roughly over 1000 patients. I have also monitored the progress long with other health recommendations such as exercise, proper diet, rest, and spinal adjustments. I have achieved with the patient amazing results of immune support and a reduction of inflammation (reducing inflammation reduces pain) in an inhospitable environment such as today. I am perhaps alone in my knowledge and patient experience and I wish to share these achievements with the world (companies will copy my vitamin volume ideas). However, I must first patent my results of my many healthy patients.

### BACKGROUND

Field of the Invention: Immune Support with Significant Reduction of Inflammation
My Inventions are Formulas of Vitamins and Immune Drinks to Support
the Immune System and to Reduce Inflammation.
Example: Formula 5 Quercetin, Zinc, D3, Calcium, Sodium, and Potassium Immune Drink
1-5000 mg Quercetin in any variation in a drink form including Quercetin Syrup Diluted with
fluid to make an Immune drink or refreshment. 250 mg used in this formula
1-200 mg of Zinc in any variation. 25 mg used in this formula
1-200 mcg of vitamin D3 of any variation. 25 mcg used in this formula
1-2000 mg of Calcium of any variation. 200 mg used in this formula
1-500 mg of Sodium Ions such as in an energy drink (160 mg per 16 oz. is commonly used and is what I use)
1-300 mg of Potassium Ions such as in energy drinks (45 mg per 16 oz. is commonly used and is what I use)
16-32 oz container is best
This Formula is best for reducing Inflammation and Immune Support.

### DESCRIPTION OF THE RELATED ART

There are 1-2 Vitamin combinations on the market with quercetin and zinc only. There are also 1-2 vitamin combinations with Elderberry and zinc combinations. I do not know if they have a patent. These are deficient in there milligram amounts for reducing inflammation and immune support. The more ingredients the more deficient they are without a real beneficial stimulation in the human body. In other words their daily recommendations are deficient. This field of vitamins in the immune support market is growing fast with the current environment we live in. However, truly there are few decent products without the real knowledge of clinical use. There are few products similar in that they don't have clinical studies and no real knowledge of how much ingredient is required per day. Also, which ingredients to be effective for inflammation reduction and immune support.

### BRIEF DESCRIPTION OF THE DIAGRAMS

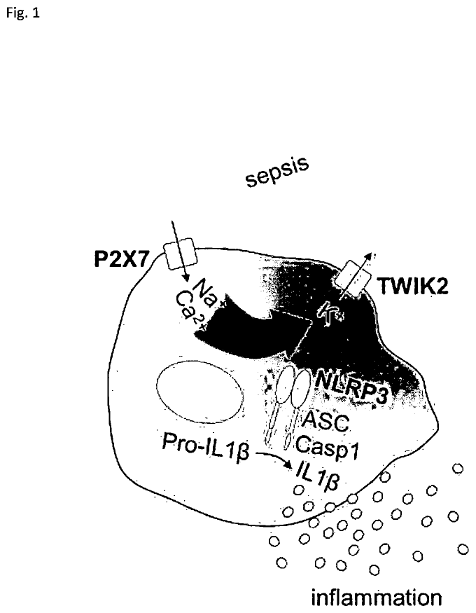

FIG. 1

I am including this diagram to describe the cellular action discovered by Dr. Jalees Rehman and Proffessor Asrar Malik of the University of Illinois in Chicago.

This diagram is a diagram of a macrophage white blood cell. This describes how White blood cells become overtly hyper excitatory with potassium and TWIK2 potassium channels. Then release Interluekin-1 Beta. Interluekin-1 Beta is associated with increasing Fibroblasts, Collagenase, and Cortisol. Inhibiting newly discovered Twik2 channels does in fact inhibit inflammation. Dr. Jalees Rehman and Proffessor Asrar Malik discovered Quinine can inhibit TWIK2 channels.

This can also be very effective in shutting down overly excited white blood cells in what has recently been called a cytokine response or storm. This is where white blood cells attack your own body because of over stimulation. Shutting down the Twik2 channels can shut down cytokine response of the compliment system. The compliment system is when a WBC punches a very tiny hole in an organ or system.

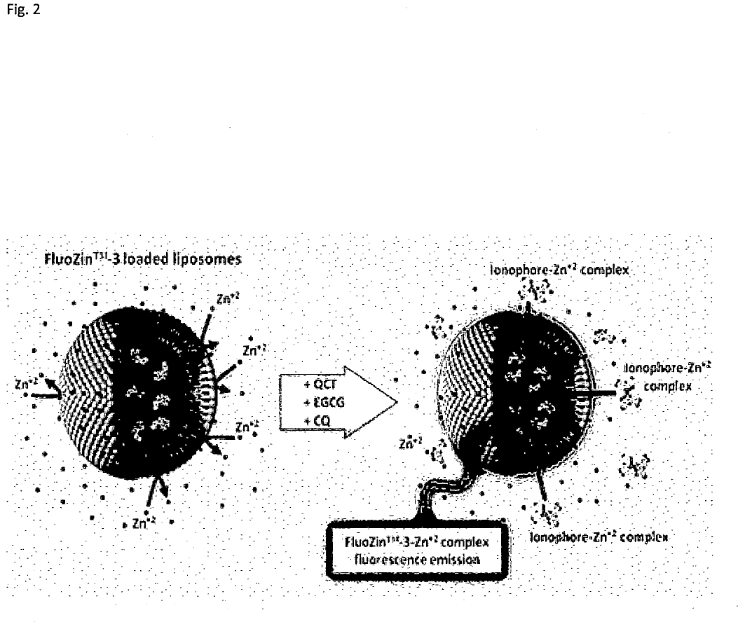

FIG. 2

This is a diagram of Zinc Ionophore Influx across the human cell plasma membrane. Plant polyphenols such as flavonoids like quercetin act as antioxidants and also carry zinc ionophore across the cell membrane. This makes the zinc a patient might take many times more effective by getting zinc inside the cell.

This action occurs when combining Quinine and zinc as well as Quercetin and zinc as well as Elderberry and Zinc. This action occurs also in other fruits and vegetables(berries, cherries, grapes, red onions, baby kale, spinach, and cherry tomatoes ect . . . . ) with high concentrations of the bioflavonoid Quercetin.

Quercetin is the active bioflavonoid in elderberry and Elderberry. There are other fruits and vegetables (berries, cherries, grapes, red onions, baby kale, spinach, and cherry tomatoes ect . . . . ) with high concentrations of the bioflavonoid Quercetin.

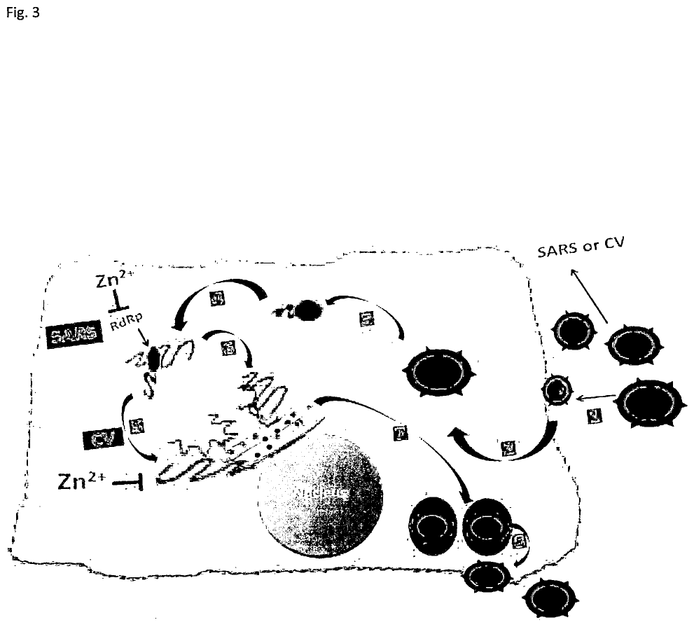

FIG. 3

This is a diagram of How Zinc inhibits the replication of viruses that try to penetrate the cell wall and cause cell death.

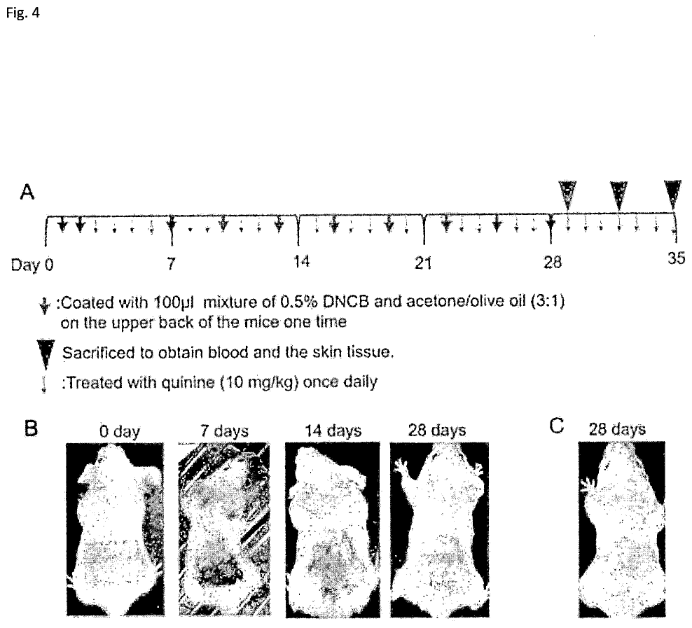

FIG. 4

This is a diagram of a study showing the anti-inflammatory properties of quinine on mice. Acetone/olive oil was placed on the mice for 4 weeks. Mice given quinine did not have a loss of skin and flesh due to the suppression of TAS2R channels in the lungs inhibited by the quinine. The inhibited TAS2R receptors cause an agonist of IGE immunoglobulin. IGE cells are associates with allergic reactions in the human body. Reducing allergic reactions in the body will inhibit inflammation. This is a great illustration of reduction of skin inflammation.

### DETAILED DESCRIPTION

Formulas listed and refer back to brief description and diagrams

Liberty Vitamins

Formula 1 Basic Immune Support with Reduction of inflammation.

1-1000 mg Quercetin of any variation (500 mg Quercetin is best for this formula).

1-50 mg of zinc of any variation (50 mg of Zinc is best for this formula).

Formula 2 Super Immune Support with Reduction of Inflammation.

1-1000 mg Quercetin of any variation (500 mg Quercetin is best for this formula).

1-50 mg of zinc of any variation (50 mg of Zinc is best for this formula).

1-50 mcg 2000 IU D3 of any variation (50 mcg 2000 IU D3 is best for this formula).

Formula 3 Super Immune Support with Reduction of Inflammation with Calcium.

1-1000 mg Quercetin of any variation (500 mg Quercetin is best for this formula).

1-50 mg of zinc of any variation (50 mg Zinc is best for this formula).

1-50 mcg 2000 IU D3 of any variation (50 mcg 2000 IU D3 is best for this formula).

1-2000 Calcium of any variation (400 mg Calcium is best for this formula).

Formula 4 Super Immune Support with Reduction of Inflammation with Calcium and extra arthritic component.

1-1000 mg Quercetin of any variation (500 mg Quercetin is best for this formula).

1-50 mg of zinc of any variation (50 mg Zinc is best for this formula).

1-50 mcg 2000 IU D3 of any variation (50 mcg 2000 IU D3 is best for this formula).

1-2000 Calcium of any variation (400 mg Calcium is best for this formula).

1-500 mg Glucosamine of any variation (500 mg Glucosamine is best for this formula).

1-1000 mg Chondroitin of any variation (150 mg Chondroitin is best for this formula).

Formula 5 Basic Immune Support with Reduction of Inflammation with Calcium.

1-1000 mg Quercetin of any variation (250 mg Quercetin is best for this formula).

1-50 mg of zinc of any variation (25 mg Zinc is best for this formula).

1-50 mcg 2000 IU D3 of any variation (25 mcg 2000 IU D3 is best for this formula).

1-2000 Calcium of any variation (200 mg Calcium is best for this formula).

Formula 6 Basic Immune Support with Reduction of Inflammation and Stomach Stability.

1-1000 mg Quercetin of any variation (250 mg Quercetin is best for this formula).

1-50 mg of zinc of any variation (25 mg Zinc is best for this formula).

1-50 mcg 2000 IU D3 of any variation (25 mcg 2000 IU D3 is best for this formula).

1-2000 Calcium of any variation (200 mg Calcium is best for this formula).

1-300 mg of Magnesium of any variation (100 mg of Magnesium is best for this formula).

Liberty Vita rains

Formula 1 Basic Immune Support with Reduction of inflammation.

1-1000 mg Quinine of any variation (500 mg Quinine is best for this formula).

1-50 mg of zinc of any variation (50 mg of Zinc is best for this formula).

Formula 2 Super Immune Support with Reduction of Inflammation.

1-1000 mg Quinine of any variation (500 mg Quinine is best for this formula).

1-50 mg of zinc of any variation (50 mg of Zinc is best for this formula).

1-50 mcg 2000 IU D3 of any variation (50 mcg 2000 IU D3 is best for this formula)

Formula 3 Super Immune Support with Reduction of Inflammation with Calcium.

1-1000 mg Quinine of any variation (500 mg Quinine is best for this formula).

1-50 mg of zinc of any variation (50 mg Zinc is best for this formula).

1-50 mcg 2000 IU D3 of any variation (50 mcg 2000 IU D3 is best for this formula).

1-2000 Calcium of any variation (400 mg Calcium is best for this formula).

Formula 4 Super Immune Support with Reduction of Inflammation with Calcium and extra arthritic component.

1-1000 mg Quinine of any variation (500 mg Quinine is best for this formula).

1-50 mg of zinc of any variation (50 mg Zinc is best for this formula).

1-50 mcg 2000 IU D3 of any variation (50 mcg 2000 IU D3 is best for this formula).

1-2000 Calcium of any variation (400 mg Calcium is best for this formula).

1-500 mg Glucosamine of any variation (500 mg Glucosamine is best for this formula).

1-1000 mg Chondroitin of any variation (150 mg Chondroitin is best for this formula).

Formula 5 Basic Immune Support with Reduction of Inflammation with Calcium.

1-1000 mg Quinine of any variation (250 mg Quinine is best for this formula).

1-50 mg of zinc of any variation (25 mg Zinc is best for this formula).

1-50 mcg 2000 IU D3 of any variation (25 mcg 2000 IU D3 is best for this formula).

1-2000 Calcium of any variation (200 mg Calcium is best for this formula).

Formula 6 Basic Immune Support with Reduction of Inflammation and Stomach Stability.

1-1000 mg Quinine of any variation (250 mg Quinine is best for this formula).

1-50 mg of zinc of any variation (25 mg Zinc is best for this formula).

1-50 mcg 2000 IU D3 of any variation (25 mcg 2000 IU D3 is best for this formula).

1-2000 Calcium of any variation (200 mg Calcium is best for this formula).

1-300 mg of Magnesium of any variation (100 mg of Magnesium is best for this formula).

Liberty Vita rains

Formula 1 Basic Immune Support with Reduction of inflammation.

500 mg Quinine

50 mg zinc

Formula 2 Super Immune Support with Reduction of inflammation.

500 mg Quinine

50 mg zinc

50 mcg 2000 IU D3

Formula 3 Super Immune Support with Reduction of inflammation with Calcium.

500 mg Quinine

50 mg zinc

50 mcg 2000 IU D3

400 mg Calcium

Formula 4 Super Immune Support with Reduction of inflammation with Calcium and extra arthritic

component

500 mg Quinine

50 mg zinc

50 mcg 2000 IU D3

400 mg Calcium

500 mg Glucosamine

150 mg Chondroitin

Formula 5 Super Immune Support with Reduction of inflammation with Calcium.

500 mg Quinine

50 mg zinc

50 mcg 2000 IU D3

400 mg Calcium

500 mg Quercetin

Formula 6 Super Immune Support with Reduction of inflammation with Calcium.

250 mg Quinine

25 mg zinc

25 mcg 2000 IU D3

200 mg Calcium

Formula 7 Gummies for basic Immune Support with Reduction of inflammation with Calcium.

250 mg Quinine

25 mg zinc

25 mcg 2000 IU D3

200 mg Calcium

Other Ingredients: Cane Syrup (sugar), Purified water, Tapioca Syrup, Pectin, Citric Acid, Sodium Citrate, Natural Flavors, Carnauba Wax.

Formula 8 Immune Support Fortified Flavored Water with Anti-inflammatory Properties. 16 FL. OZ.

250 mg Quercetin

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 9 Immune Support Fortified Flavored Water with Anti-inflammatory Properties. 16 FL. OZ.

250 mg Quinine

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 10 Immune Support Fortified Flavored Water with Anti-inflammatory Properties. 16 FL. OZ.

250 mg Quercetin

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 11 Immune Support Fortified Flavored Water with Anti-inflammatory Properties. 16 FL. OZ.

500 mg Elderberry

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 12 Immune Support Fortified Flavored Water with Anti-inflammatory Properties. 16 FL. OZ.

500 mg Elderberry

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 13 Immune Support Fortified Flavored Water with Anti-inflammatory Properties. 16 FL. OZ.

250 mg Quinine

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 14 Immune Support Fortified Flavored Water with Anti-inflammatory Properties. 16 FL. OZ.

250 mg Quinine

25 mg Zinc

25 mcg 2000 IU D3

50 mg Calcium (Calcium Chloride)

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

The basic patent is for Super Vitamin Grouping for reducing inflammation and stimulating the body's immune system as well as reducing arthritis. There are several ingredients as listed below for the perfect blend I have recommended for my patients:

1-1000 mg Quinine of any variation (250 mg or 500 mg Quinine used for these formulas).

1-50 mg of Zinc of any variation (25 mg or 50 mg Zinc used for these formulas).

1-50 mcg 2000 IU D3 of any variation (25 mcg or 50 mcg 2000 IU D3 used for these formulas).

1-2000 Calcium of any variation (200 mg or 400 mg Calcium used for these formulas).

1-500 mg Glucosamine of any variation (500 mg Glucosamine is best for this formula).

1-1000 mg Chondroitin of any variation (150 mg Chondroitin is best for this formula).

1-1000 mg Quercetin of any variation (250 mg or 500 mg Quercetin used for these formulas).

1-2000 mg Elderberry of any variation (500 mg or 1000 mg Elderberry used for these formulas).

1-500 mg of Magnesium of any variation (100 mg Magnesium used for these formulas).

Liberty Vita rains

Formula 1 Basic Immune Support with reduction of inflammation.

1-2000 mg Elderberry of any variation (1000 mg Elderberry is best for this formula)

1-50 mg Zinc of any variation (50 mg zinc is best for this formula) Formula 2 Super Immune Support with reduction of inflammation.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

Formula 3 Super Immune Support with reduction of inflammation with Calcium.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

Formula 4 Super Immune Support with reduction of inflammation with Calcium and Arthritic component.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-500 mg Glucosamine of any variation (500 mg of Glucosa mine is best for this formula)

1-1000 mg of Chondroitin of any variation (150 mg of Chondroitin is best for this formula)

Formula 5 Super Immune Support with reduction of inflammation and Calcium and Extra Immune Support.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Quercetin of any variation (500 mg Quercetin is best for this formula)

Formula 6 Basic Immune Support with reduction of inflammation.

1-2000 mg Elderberry of any variation (500 mg Elderberry is best for this formula)

1-50 mg Zinc of any variation (25 mg zinc is best for this formula)

1-50 mcg or 1-2000 IU of D3 of any variation (25 mcg or 2000 IU of D3 is best for this formula)

1-2000 mg of Calcium of any variation (200 mg Calcium is best for this formula)

Formula 7 Super Immune Support with reduction of inflammation and Calcium and Extra times 2 Immune Support.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Quercetin of any variation (500 mg Quercetin is best for this formula)

1-1000 mg of Quinine of any variation (250 mg Quinine is best for this formula)

Formula 8 Super Immune Support with reduction of inflammation and Calcium and Extra times 2 Immune Support.

1-2000 mg Elderberry of any variation (500 mg Elderberry is best for this formula)

1-50 mg Zinc of any variation (25 mg zinc is best for this formula)

1-50 mcg or 1-2000 IU of D3 of any variation (25 mcg or 2000 IU of D3 is best for this formula)

1-2000 mg of Calcium of any variation (200 mg Calcium is best for this formula)

1-1000 mg of Quercetin of any variation (250 mg Quercetin is best for this formula)

1-1000 mg of Quinine of any variation (125 mg Quinine is best for this formula)

Liberty Vita rains

Formula 1 Basic Immune Support with reduction of inflammation.

1-1000 mg Quinine of any variation (500 mg Quinine is best for this formula)

1-50 mg Zinc of any variation (50 mg zinc is best for this formula)

1-1000 mg of Vitamin C of any variation (200 mg Vitamin C is best for this formula)

Formula 2 Super Immune Support with reduction of inflammation.

1-1000 mg Quinine of any variation (500 mg Quinine is best for this formula)

1-50 mg Zinc of any variation (50 mg zinc is best for this formula)

1-50 mcg or 1-2000 IU of D3 of any variation (50 mcg or 2000 IU of D3 is best for this formula)

1-1000 mg of Vitamin C of any variation (200 mg Vitamin C is best for this formula) Formula 3 Super Immune Support with reduction of inflammation with Calcium.

1-1000 mg Quinine of any variation (500 mg Quinine is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Vitamin C of any variation (200 mg Vitamin C is best for this formula)

Formula 4 Super Immune Support with reduction of inflammation with Calcium and Arthritic component.

1-1000 mg Quinine of any variation (500 mg Quinine is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-500 mg Glucosamine of any variation (500 mg of Glucosamine is best for this formula)

1-1000 mg of Chondroitin of any variation (150 mg of Chondroitin is best for this formula)

1-1000 mg of Vitamin C of any variation (200 mg Vitamin C is best for this formula)

Formula 5 Super Immune Support with reduction of inflammation and Calcium and Extra Immune Support.

1-1000 mg Quinine of any variation (500 mg Quinine is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Quercetin of any variation (500 mg Quercetin is best for this formula)

1-1000 mg of Vitamin C of any variation (200 mg Vitamin C is best for this formula)

Formula 6 Basic Immune Support with reduction of inflammation.

1-1000 mg Quinine of any variation (250 mg Quinine is best for this formula)

1-50 mg Zinc of any variation (25 mg zinc is best for this formula)

1-50 mcg or 1-2000 IU of D3 of any variation (25 mcg or 2000 IU of D3 is best for this formula)

1-2000 mg of Calcium of any variation (200 mg Calcium is best for this formula)

1-1000 mg of Vitamin C of any variation (200 mg Vitamin C is best for this formula)

Liberty Vitamins

Formula 1 Basic Immune Support with reduction of inflammation.

1-1000 mg Quercetin of any variation (500 mg Quercetin is best for this formula)

1-50 mg Zinc of any variation (50 mg zinc is best for this formula)

1-1000 mg of Vitamin C of any variation (200 mg Vitamin C is best for this formula)

Formula 2 Super Immune Support with reduction of inflammation.

1-1000 mg Quercetin of any variation (500 mg Quercetin is best for this formula)

1-50 mg Zinc of any variation (50 mg zinc is best for this formula)

1-50 mcg or 1-2000 IU of D3 of any variation (50 mcg or 2000 IU of D3 is best for this formula)

1-1000 mg of Vitamin C of any variation (200 mg Vitamin C is best for this formula)

Formula 3 Super Immune Support with reduction of inflammation with Calcium.

1-1000 mg Quercetin of any variation (500 mg Quercetin is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Vitamin C of any variation (200 mg Vitamin C is best for this formula)

Formula 4 Super Immune Support with reduction of inflammation with Calcium and Arthritic component.

1-1000 mg Quercetin of any variation (500 mg Quercetin is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-500 mg Glucosamine of any variation (500 mg of Glucosamine is best for this formula)

1-1000 mg of Chondroitin of any variation (150 mg of Chondroitin is best for this formula)

1-1000 mg of Vitamin C of any variation (200 mg Vitamin C is best for this formula)

Formula 5 Super Immune Support with reduction of inflammation and Calcium and Extra Immune Support.

1-1000 mg Quercetin of any variation (500 mg Quercetin is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-2000 mg of Elderberry of any variation (1000 mg Elderberry is best for this formula)

1-1000 mg of Vitamin C of any variation (200 mg Vitamin C is best for this formula)

Formula 6 Basic Immune Support with reduction of inflammation.

1-1000 mg Quercetin of any variation (250 mg Quercetin is best for this formula)

1-50 mg Zinc of any variation (25 mg zinc is best for this formula)

1-50 mcg or 1-2000 IU of D3 of any variation (25 mcg or 2000 IU of D3 is best for this formula)

1-2000 mg of Calcium of any variation (200 mg Calcium is best for this formula)

1-1000 mg of Vitamin C of any variation (200 mg Vitamin C is best for this formula)

Liberty Vita rains

Formula 1 Basic Immune Support with reduction of inflammation.

1-2000 mg Elderberry of any variation (1000 mg Elderberry is best for this formula)

1-50 mg Zinc of any variation (50 mg zinc is best for this formula)

1-1000 mg of Vitamin C of any variation (200 mg Vitamin C is best for this formula)

Formula 2 Super Immune Support with reduction of inflammation.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-1000 mg of Vitamin C of any variation (200 mg Vitamin C is best for this formula)

Formula 3 Super Immune Support with reduction of inflammation with Calcium.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Vitamin C of any variation (200 mg Vitamin C is best for this formula)

Formula 4 Super Immune Support with reduction of inflammation with Calcium and Arthritic component.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-500 mg Glucosamine of any variation (500 mg of Glucosamine is best for this formula)

1-1000 mg of Chondroitin of any variation (150 mg of Chondroitin is best for this formula)

1-1000 mg of Vitamin C of any variation (200 mg Vitamin C is best for this formula)

Formula 5 Super Immune Support with reduction of inflammation and Calcium and Extra Immune Support.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Quercetin of any variation (500 mg Quercetin is best for this formula)

1-1000 mg of Vitamin C of any variation (200 mg Vitamin C is best for this formula)

Formula 6 Basic Immune Support with reduction of inflammation.

1-2000 mg Elderberry of any variation (500 mg Elderberry is best for this formula)

1-50 mg Zinc of any variation (25 mg zinc is best for this formula)

1-50 mcg or 1-2000 IU of D3 of any variation (25 mcg or 2000 IU of D3 is best for this formula)

1-2000 mg of Calcium of any variation (200 mg Calcium is best for this formula)

1-1000 mg of Vitamin C of any variation (200 mg Vitamin C is best for this formula)

Formula 7 Super Immune Support with reduction of inflammation and Calcium and Extra times 2 Immune Support.

1-2000 mg Elderberry of any variation (1000 mg Elderberry is best for this formula)

1-50 mg Zinc of any variation (50 mg zinc is best for this formula)

1-50 mcg or 1-2000 IU of D3 of any variation (50 mcg or 2000 IU of D3 is best for this formula)

1-2000 mg of Calcium of any variation (400 mg Calcium is best for this formula)

1-1000 mg of Quercetin of any variation (500 mg Quercetin is best for this formula)

1-1000 mg of Quinine of any variation (250 mg Quinine is best for this formula)

1-1000 mg of Vitamin C of any variation (200 mg Vitamin C is best for this formula)

Formula 8 Super Immune Support with reduction of inflammation and Calcium and Extra times 2 Immune Support.

1-2000 mg Elderberry of any variation (500 mg Elderberry is best for this formula)

1-50 mg Zinc of any variation (25 mg zinc is best for this formula)

1-50 mcg or 1-2000 IU of D3 of any variation (25 mcg or 2000 IU of D3 is best for this formula)

1-2000 mg of Calcium of any variation (200 mg Calcium is best for this formula)

1-1000 mg of Quercetin of any variation (250 mg Quercetin is best for this formula)

1-1000 mg of Quinine of any variation (125 mg Quinine is best for this formula)

1-1000 mg of Vitamin C of any variation (100 mg Vitamin C is best for this formula)

Liberty Vita rains

Formula 1 Basic Immune Support with Reduction of inflammation.

500 mg Quinine

50 mg zinc

200 mg Vitamin C

Formula 2 Super Immune Support with Reduction of inflammation.

500 mg Quinine

50 mg zinc

50 mcg 2000 IU D3

200 mg Vitamin C

Formula 3 Super Immune Support with Reduction of inflammation with Calcium.

500 mg Quinine

50 mg zinc

50 mcg 2000 IU D3

400 mg Calcium

200 mg Vitamin C

Formula 4 Super Immune Support with Reduction of inflammation with Calcium and extra arthritic component

500 mg Quinine

50 mg zinc

50 mcg 2000 IU D3

400 mg Calcium

500 mg Glucosamine

150 mg Chondroitin

200 mg Vitamin C

Formula 5 Super Immune Support with Reduction of inflammation with Calcium.

500 mg Quinine

50 mg zinc

50 mcg 2000 IU D3

400 mg Calcium

500 mg Quercetin

200 mg Vitamin C

Formula 6 Basic Immune Support with Reduction of inflammation with Calcium.

250 mg Quinine

25 mg zinc

25 mcg 2000 IU D3

200 mg Calcium

200 mg Vitamin C

Formula 7 Gummies for basic Immune Support with Reduction of inflammation with Calcium.

250 mg Quinine

25 mg zinc

25 mcg 2000 IU D3

200 mg Calcium

200 mg Vitamin C

Other Ingredients: Cane Syrup (sugar), Purified water, Tapioca Syrup, Pectin, Citric Acid, Sodium Citrate, Natural Flavors, Carnauba Wax.

Formula 8 Immune Support Fortified Flavored Water with Anti-inflammatory Properties. 16 FL. OZ.

250 mg Quercetin

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 9 Immune Support Fortified Flavored Water with Anti-inflammatory Properties. 16 FL. OZ.

250 mg Quinine

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 10 Immune Support Fortified Flavored Water with Anti-inflammatory Properties. 16 FL. OZ.

250 mg Quercetin

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 11 Immune Support Fortified Flavored Water with Anti-inflammatory Properties. 16 FL. OZ.

1000 mg Elderberry

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 12 Immune Support Fortified Flavored Water with Anti-inflammatory Properties. 16 FL. OZ.

1000 mg Elderberry

25 mg Zinc

25 mcg 2000 IU D3

50 mg Calcium (Calcium Chloride) 200 mg Vitamin C

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 13 Immune Support Fortified Flavored Water with Anti-inflammatory Properties. 16 FL. OZ.

250 mg Quercetin

1000 mg Elderberry

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 14 Immune Support Fortified Flavored Water with Anti-inflammatory Properties. 16 FL. OZ.

250 mg Quinine

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 15 Immune Support Fortified Flavored Water with Anti-inflammatory Properties. 16 FL. OZ.

250 mg Quercetin

500 mg Elderberry

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 16 Immune Support Fortified Flavored Water with Anti-inflammatory Properties. 16 FL. OZ.

250 mg Quercetin

500 mg Elderberry

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

Formula 17 Immune Support Fortified Flavored Water with Anti-inflammatory Properties. 16 FL. OZ.

500 mg Quercetin

1000 mg Elderberry

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 18 Immune Support Fortified Flavored Water with Anti-inflammatory Properties. 16 FL. OZ.

500 mg Quercetin

1000 mg Elderberry

500 mg Quinine

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Salt, Magnesium, Calcium Chloride, Calcium Disodium EDTA.

Formula 19 Immune Support Fortified Flavored Water with Anti-inflammatory Properties. 16 FL. OZ.

500 mg Quercetin

1000 mg Elderberry

500 mg Quinine

25 mg Zinc

25 mcg 2000 IU D3

50 mg Calcium (Calcium Chloride)

200 mg Vitamin C

Other Ingredients: Fruit Juice Blend, Filtered Water, Raspberry Juice Concentrate, Potassium Benzoate, Cholecalciferol (D3), Cyanocobalamin (B12), Pyridoxine Hydrochloride (B6), Monopotassium Phosphate, Salt, Magnesium, Calcium Chloride, Calcium Disodium EDTA.

The basic patent is for Super Vitamin Grouping for reducing inflammation and stimulating the body's immune system as well as reducing arthritis. There are several ingredients as listed below for the perfect blend I have recommended for my patients:

1-1000 mg Quinine of any variation (250 mg or 500 mg Quinine used for these formulas).

1-50 mg of Zinc of any variation (25 mg or 50 mg Zinc used for these formulas).

1-50 mcg 2000 IU D3 of any variation (25 mcg or 50 mcg 2000 IU D3 used for these formulas).

1-2000 Calcium of any variation (200 mg or 400 mg Calcium used for these formulas).

1-500 mg Glucosa mine of any variation (500 mg Glucosamine is best for this formula).

1-1000 mg Chondroitin of any variation (150 mg Chondroitin is best for this formula).

1-1000 mg Quercetin of any variation (250 mg or 500 mg Quercetin used for these formulas).

1-2000 mg Elderberry of any variation (500 mg or 1000 mg Elderberry used for these formulas).

1-500 mg of Magnesium of any variation (100 mg Magnesium used for these formulas).

1 mg-1000 mg of Vitamin C (200 mg of Vitamin C used in these formulas).

Monopotassium Phosphate, Potassium Benzoate, and Salt (Sea salt Preferable) used in these formulas

The basic patent is for Super vitamin grouping for reducing inflammation and stimulating the body's immune system as well as reducing arthritis. There are several ingredients as listed below for the perfect blend for achieving health that I have recommended and monitored for my patient's:

500 mg-1000 mg Quinine (250 mg or 500 mg Quinine used in these formulas)

25-50 mg Zinc of (25 mg or 50 mg zinc is best for this formula)

25-50 mcg or 1-2000 IU of D3 (25 mcg or 50 mcg or 2000 IU of D3 is best for this formula)

200-1000 mg of Calcium of any variation (200 mg or 400 mg Calcium is best for this formula)

Same formula above with extra added arthritic component

250-1000 mg Glucosamine (500 mg of Glucosamine is best for this formula)

150-1250 mg of Chondroitin (150 mg of Chondroitin is best for this formula)

Same formula above with extra added immune booster and inflammation reduction component

250-1000 mg of Quercetin (250 mg or 500 mg Quercetin is best for this formula)

250-2000 mg Elderberry (500 mg or 1000 mg Elderberry is best for this formula)

200-1000 mg of Vitamin C (200 mg Vitamin C is best for this formula)

Any Immune Drink with Quercetin

Formula 1 Quercetin Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1 Oz.-2 gallon container or any variation for sale

6 Oz.-16 Oz. container is best

Formula 2 Quercetin and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1 Oz.-2 gallon container or any variation for sale

6 Oz.-16 Oz. container is best

Formula 3 Quercetin and Zinc Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-1000 mg of Vitamin C of any variation (200 mg is best for this formula)

1 Oz.-2 gallon container or any variation for sale

6 Oz.-16 Oz. container is best

Formula 4 Quercetin, Zinc, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 5 Quercetin, Zinc, and D3 Immune Drink 1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-200 mcg of vitamin D3 of any variation (25-50 mcg is best for this formula)

1 Oz.-2 gallon container or any variation for sale

6 Oz.-16 Oz. container is best

Formula 6 Quercetin, Zinc, D3, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1 Oz.-2 gallon container or any variation for sale

6 Oz.-16 oz. container is best

Formula 7 Quercetin, Zinc, D3, and Calcium Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 oz. container is best

Formula 8 Quercetin, Zinc, D3, Calcium, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1 Oz.-2 gallon container or any variation for sale

6 Oz.-16 oz. container is best

Formula 9 Quercetin, Zinc, D3, Calcium, Sodium, and Potassium Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 oz. container is best

Formula 10 Quercetin, Zinc, D3, Calcium, Sodium, Potassium, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz. -16 Oz. container is best

Any Immune Drink with Elderberry

Formula 1 Elderberry Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1 Oz.-2 gallon container or any variation for sale

6 Oz. -16 Oz. container is best Formula 2 Elderberry and Vitamin C Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1 Oz.-2 gallon container or any variation for sale

6 Oz. -16 Oz. container is best

Formula 3 Elderberry and Zinc Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-1000 mg of Vitamin C of any variation (200 mg is best for this formula)

1 Oz.-2 gallon container or any variation for sale

6 Oz.-16 Oz. container is best

Formula 4 Elderberry, Zinc, and Vitamin C Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 5 Elderberry, Zinc, and D3 Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1 Oz.-2 gallon container or any variation for sale

6 Oz.-16 Oz. container is best

Formula 6 Elderberry, Zinc, D3, and Vitamin C Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1 Oz.-2 gallon container or any variation for sale

6 Oz.-16 Oz. container is best

Formula 7 Elderberry, Zinc, D3, and Calcium Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vita min of any variation (50 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 Oz. container is best

Formula 8 Elderberry, Zinc, D3, Calcium, and Vitamin C Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1 Oz.-2 gallon container or any variation for sale

6 Oz.-16 Oz. container is best

Formula 9 Elderberry, Zinc, D3, Calcium, Sodium, and Potassium Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Formula 10 Elderberry, Zinc, D3, Calcium, Sodium, Potassium, and Vitamin C Immune Drink

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 oz. container is best

Any Immune Drink with Quercetin

Formula 1 Quercetin and Elderberry Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1 Oz.-2 gallon container or any variation for sale

6 Oz.-16 Oz. container is best

Formula 2 Quercetin, Elderberry, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1 Oz.-2 gallon container or any variation for sale

6 Oz.-16 Oz. container is best

Formula 3 Quercetin, Elderberry, and Zinc Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-1000 mg of Vitamin C of any variation (200 mg is best for this formula)

1 Oz.-2 gallon container or any variation for sale

6 Oz.-16 Oz. container is best

Formula 4 Quercetin, Elderberry, Zinc, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 5 Quercetin, Elderberry, Zinc, and D3 Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1 Oz.-2 gallon container or any variation for sale

6 Oz.-16 Oz. container is best

Formula 6 Quercetin, Elderberry, Zinc, D3, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1 Oz.-2 gallon container or any variation for sale

6 Oz.-16 Oz. container is best

Formula 7 Quercetin, Elderberry, Zinc, D3, and Calcium Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 Oz. container is best

Formula 8 Quercetin, Elderberry, Zinc, D3, Calcium, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1 Oz.-2 gallon container or any variation for sale

6 Oz.-16 Oz. container is best

Formula 9 Quercetin, Elderberry, Zinc, D3, Calcium, Sodium, and Potassium Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 oz. container is best

Formula 10 Quercetin, Elderberry, Zinc, D3, Calcium, Sodium, Potassium, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 oz. container is best

Any Immune Drink with Quercetin

Formula 1 Quercetin, Quinine, and Elderberry Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1 Oz.-2 gallon container or any variation for sale

6 Oz.-16 Oz. container is best

Formula 2 Quercetin, Quinine, Elderberry, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1 Oz.-2 gallon container or any variation for sale

6 Oz.-16 Oz. container is best

Formula 3 Quercetin Quinine, Elderberry, and Zinc Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-1000 mg of Vitamin C of any variation (200 mg is best for this formula)

1 Oz.-2 gallon container or any variation for sale

6 Oz.-16 Oz. container is best

Formula 4 Quercetin, Quinine, Elderberry, Zinc, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 5 Quercetin, Quinine, Elderberry, Zinc, and D3 Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-200 mcg of vitamin D3 of any variation (25-50 mcg is best for this formula)

1 Oz.-2 gallon container or any variation for sale

6 Oz.-16 Oz. container is best

Formula 6 Quercetin, Quinine, Elderberry, Zinc, D3, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1 Oz.-2 gallon container or any variation for sale

6 Oz.-16 Oz. container is best

Formula 7 Quercetin, Quinine, Elderberry, Zinc, D3, and Calcium Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 Oz. container is best

Formula 8 Quercetin, Quinine Elderberry, Zinc, D3, Calcium, and Vitamin C Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1 Oz.-2 gallon container or any variation for sale

6 Oz.-16 Oz. container is best

Formula 9 Quercetin, Quinine, Elderberry, Zinc, D3, Calcium, Sodium, and Potassium Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Formula 10 Quercetin, Quinine, Elderberry, Zinc, D3, Calcium, Sodium, Potassium, and Vitamin C

Immune Drink

1-5000 mg Quercetin Immune drink in any variation in a drink form. This Includes Quercetin Syrup diluted with fluid or water to make an Immune drink or refreshment. Also includes Quercetin powder diluted with water or fluid to make an immune drink or refreshment.

100 mg-1000 mg Quercetin (500 mg is best per container)

1-5000 mg Elderberry Immune drink in any variation in a drink form. This Includes Elderberry Syrup diluted with fluid or water to make an Immune drink or refreshment. Also includes Elderberry powder diluted with water or fluid to make an immune drink or refreshment.

100 mg-2000 mg Elderberry (1000 mg is best per container)

1-5000 mg Quinine Immune drink in any variation in a drink form. This Includes Quinine Syrup diluted with fluid or water to make an Immune drink or refreshment. Also includes Quinine powder diluted with water or fluid to make an immune drink or refreshment.

100 mg-1000 mg Quinine (500 mg is best per container)

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 oz. container is best

There are many foods with the high values of Quercetin that can replace Elderberry and the capacity to be effective. These foods are any berry extract especially(1) Blueberries, (2) Strawberries, (3) Phytolacca, (4) Acai, just to name a few. Quercetin also has high values in Cherries and grapes. Quercetin also has high values in red onions, Kale, Cherry Tomatoes, and Spinach. The goal is to get enough milligrams through extract of these foods to be effective. This could mean values of 2000-5000 my per drink or vitamin product.

We will use cherries, blueberries, berries, and grapes.

We will also use red onions, kale, Cherry Tomatoes and spinach.

Formula 1 Basic Immune Support with reduction of Inflammation.

1-5000 mg berries, cherries, or grape extract of any variation (1000-2000 mg of berries, cherries, or grape extract is best for this formula)

Formula 2 Basic Immune Support with reduction of Inflammation.

1-5000 mg berries, cherries, or grape extract of any variation (1000-2000 mg of berries, cherries, or grape extract is best for this formula

1-50 mg zinc of any variation (50 mg of zinc is best for this formula)

Formula 3 super Immune Support with reduction of Inflammation.

1-5000 mg berries, cherries, or grape extract of any variation (1000-2000 mg of berries, cherries, or grape extract is best for this formula)

1-50 mg zinc of any variation (50 mg of zinc is best for this formula)

1-1000 mg of Vitamin C of any Variation (200 mg of Vitamin C for this formula

Formula 4 super Immune Support with reduction of Inflammation.

1-5000 mg berries, cherries, or grape extract of any variation (1000-2000 mg of berries, cherries, or grape extract is best for this formula)

1-50 mg zinc of any variation (50 mg of zinc is best for this formula)

1-1000 mg of Vitamin C of any Variation (200 mg of Vitamin C for this formula)

1-50 mcg or 1-2000 IU of D3 of any Variation (50 mcg or 2000 IU used for this formula)

Formula 5 Super Immune Support with reduction of Inflammation and Calcium.

1-5000 mg berries, cherries, or grape extract of any variation (1000-2000 mg of berries, cherries, or grape extract is best for this formula)

1-50 mg zinc of any variation (50 mg of zinc is best for this formula)

1-1000 mg of Vitamin C of any Variation (200 mg of Vitamin C for this formula)

1-50 mcg or 1-2000 IU of D3 of any Variation (50 mcg or 2000 IU used for this formula)

1-2000 mg of Calcium of any variation (400 mg of Calcium used for this formula)

Formula 6 super Immune Support with reduction of Inflammation with Calcium and extra immune support.

1-5000 mg berries, cherries, or grape extract of any variation (1000-2000 mg of berries, cherries, or grape extract is best for this formula)

1-50 mg zinc of any variation (50 mg of zinc is best for this formula)

1-1000 mg of Vitamin C of any Variation (200 mg of Vitamin C for this formula)

1-50 mcg or 1-2000 IU of D3 of any Variation (50 mcg or 2000 IU used for this formula)

1-2000 mg of Calcium of any variation (400 mg of Calcium used for this formula)

1-1000 mg Quercetin of any variation 500 mg of Quercetin for this formula)

Formula 7 super Immune Support with reduction of Inflammation with Calcium and extra times 2 immune support.

1-5000 mg berries, cherries, or grape extract of any variation (1000-2000 mg of berries, cherries, or grape extract is best for this formula)

1-50 mg zinc of any variation (50 mg of zinc is best for this formula)

1-1000 mg of Vitamin C of any variation (200 mg of Vita min C for this formula)

1-50 mcg or 1-2000 IU of D3 of any Variation (50 mcg or 2000 IU used for this formula

1-2000 mg of Calcium of any variation (400 mg of Calcium used for this formula)

1-1000 mg Quercetin of any variation (500 mg of Quercetin for this formula)

1-1000 mg of quinine of any variation (250 mg of Quinine used for this formula)

Formula 8 super Immune Support with reduction of Inflammation with Calcium, immune support and extra arthritic component.

1-50 mg zinc of any variation (50 mg of zinc is best for this formula)

1-1000 mg of Vitamin C of any Variation (200 mg of Vitamin C for this formula)

1-50 mcg or 1-2000 IU of D3 of any Variation (50 mcg or 2000 IU used for this formula)

1-2000 mg of Calcium of any variation (400 mg of Calcium used for this formula)

1-500 mg of glucosamine of any variation (500 mg of Glucosamine for this formula)

1-1000 mg of Chondroitin of any variation (150 mg of Chondroitin for this formula)

Formula 1 Immune Support Fortified Flavor water with anti-Inflammatory properties. 16 Fl. OZ.

500-2000 mg berries, cherries, or grape extract of any variation (1000 mg used)

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 2 Immune Support Fortified Flavor water with anti-Inflammatory properties. 16 Fl. OZ.

500-2000 mg berries, cherries, or grape extract of any variation (1000 mg used) 25 mg of Zinc

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 3 Immune Support Fortified Flavor water with anti-Inflammatory properties. 16 Fl. OZ.

500-2000 mg berries, cherries, or grape extract of any variation (1000 mg used) 25 mg of Zinc 25 mcg or 1000 IU vitamin D3

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 4 Immune Support Fortified Flavor water with anti-Inflammatory properties. 16 Fl. OZ.

500-2000 mg berries, cherries, or grape extract of any variation (1000 mg used) 25 mg of Zinc

25 mcg or 1000 IU vitamin D3

50 mg of Calcium (Calcium Chloride)

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 5 Immune Support Fortified Flavor water with anti-Inflammatory properties. 16 Fl. OZ.

500-2000 mg berries, cherries, or grape extract of any variation (1000 mg used)

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 6 Immune Support Fortified Flavor water with anti-Inflammatory properties. 16 Fl. OZ.

500-2000 mg berries, cherries, or grape extract of any variation (1000 mg used)

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Salt, Magnesium, Calcium Chloride, and Calcium Disodium EDTA.

Formula 7 Immune Support Fortified Flavor water with anti-Inflammatory properties. 16 Fl. OZ.

500-2000 mg berries, cherries, or grape extract of any variation (1000 mg used)

250 mg of Quercetin

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 8 Immune Support Fortified Flavor water with anti-Inflammatory properties. 16 Fl. OZ.

500-2000 mg berries, cherries, or grape extract of any variation (1000 mg used)

250 mg of Quercetin

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Salt, Magnesium, Calcium Chloride, and Calcium Disodium EDTA.

Formula 9 Immune Support Fortified Flavor water with anti-Inflammatory properties. 16 Fl. OZ.

500-2000 mg berries, cherries, or grape extract of any variation (1000 mg used)

250 mg of Quercetin

250 mg of Quinine

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 10 Immune Support Fortified Flavor water with anti-Inflammatory properties. 16 Fl. OZ.

500-2000 mg berries, cherries, or grape extract of any variation (1000 mg used)

250 mg of Quercetin

250 mg of Quinine

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Salt, Magnesium, Calcium Chloride, and Calcium Disodium EDTA.

Formula 11 Immune Support Fortified Flavor water with anti-Inflammatory properties. 16 Fl. OZ.

500-2000 mg berries, cherries, or grape extract of any variation (1000 mg used)

500 mg of Quercetin

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Salt, Magnesium, Calcium Chloride, and Calcium Disodium EDTA.

Formula 12 Immune Support Fortified Flavor water with anti-Inflammatory properties. 16 Fl. OZ.

500-2000 mg berries, cherries, or grape extract of any variation (1000 mg used)

500 mg of Quercetin

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Formula 13 Immune Support Fortified Flavor water with anti-Inflammatory properties. 16 Fl. OZ.

Extra Strength

500-2000 mg berries, cherries, or grape extract of any variation (1000 mg used)

500 mg of Quercetin

500 mg of Quinine

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Salt, Magnesium, Calcium Chloride, and Calcium Disodium EDTA.

Formula 14 Immune Support Fortified Flavor water with anti-Inflammatory properties. 16 Fl. OZ.

Extra Strength

500-2000 mg berries, cherries, or grape extract of any variation (1000 mg used)

500 mg of Quercetin

500 mg of Quinine

25 mg of Zinc

25 mcg or 1000 IU vitamin D3

50 mg of Calcium (Calcium Chloride)

200 mg of Vitamin C

Formula 15 Immune Support Fortified Flavor water with anti-Inflammatory properties. 16 Fl. OZ.

Extra Strength

500-2000 mg berries, cherries, or grape extract of any variation (1000 mg used)

500 mg of Quercetin

500 mg of Quinine

25 mg of Zinc

25 mcg or 1000 IU vitamin D3

50 mg of Calcium (Calcium Chloride)

200 mg of Vitamin C

Other Ingredients: Fruit juice Blend, Filtered water, Berry Juice concentrate, Potassium Benzoate, Cholecalciferol (D3), Cyanocobalamin (B12), Pyroxene Hydrochloride B6, Monopotassium Phosphate, Salt, Magnesium, Calcium Chloride, Calcium Disodium EDTA.

Any Immune Drink with Berries, Cherries, or Grape extract of any variation (Value high Quecertin levels).

Formula 1 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1 Oz.-2 gallon container or any variation for sale

6 Oz.-16 Oz. container is best

Formula 2 Berries, Cherries, or Grape extract Immune Drink

1-5000 mg Berries, Cherries, or Grape extract Immune drink in any variation in a drink form. This

Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1 Oz.-2 gallon container or any variation for sale

6 Oz.-16 Oz. container is best

Formula 3 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-1000 mg of Vitamin C of any variation (200 mg is best for this formula)

1 Oz.-2 gallon container or any variation for sale

6 Oz.-16 Oz. container is best

Formula 4 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or flu id to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 5 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1 Oz.-2 gallon container or any variation for sale

6 Oz.-16 Oz. container is best

Formula 6 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1 Oz.-2 gallon container or any variation for sale

6 Oz.-16 Oz. container is best

Formula 7 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 Oz. container is best

Formula 8 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-1000 mg of Vitamin C of any variation (200 mg is best for this formula)

1 Oz.-2 gallon container or any variation for sale

6 Oz.-16 Oz. container is best

Formula 9 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Formula 10 Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1-1000 mg of Vitamin C of any variation (200 mg is best for this formula)

1 Oz.-2 gallon container or any variation for sale

6 Oz.-16 Oz. container is best

Any Immune Drink with Quercetin and Berries, Cherries, or Grape extract of any variation (Value high Quercetin levels).

Formula 1 Quercetin and Berries, Cherries, or Grape extract Immune Drink 1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1 Oz.-2 gallon container or any variation for sale

6 Oz.-16 Oz. container is best

Formula 2 Quercetin and Berries, Cherries, or Grape extract Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This

Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1 Oz.-2 gallon container or any variation for sale

6 Oz.-16 Oz. container is best

Formula 3 Quercetin and Berries, Cherries, or Grape extract Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 4 Quercetin and Berries, Cherries, or Grape extract Immune Drink 1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-1000 mg of Vitamin C of any variation (200 mg is best for this formula)

1-200 mcg of vitamin D3 of any variation (25-50 mcg is best for this formula)

1 Oz.-2 gallon container or any variation for sale

6 Oz.-16 Oz. container is best

Formula 5 Quercetin and Berries, Cherries, or Grape extract Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-1000 mg of Vitamin C of any variation (200 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 Oz. container is best

Formula 6 Quercetin and Berries, Cherries, or Grape extract Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-200 mg of Zinc Vitamin of any variation (50 mg is best for this formula)

1-1000 mg of Vitamin C of any variation (200 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Formula 7 Quercetin, Quinine, and Berries, Cherries, or Grape extract Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-5000 mg Quinine Immune drink in any variation in a drink form. This Includes Quinine Syrup diluted with fluid or water to make an Immune drink or refreshment. Also includes Quinine extract powder diluted with water or fluid to make an immune drink or refreshment.

100 mg-1000 mg Quinine is best per container

1-200 mg of Zinc Vitamin of any variation (50 mg is best for this formula)

1-1000 mg of Vitamin C of any variation (200 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Formula 8 Quinine and Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

1-5000 mg Quinine Immune drink in any variation in a drink form. This Includes Quinine Syrup diluted with fluid or water to make an Immune drink or refreshment. Also includes Quinine extract powder diluted with water or fluid to make an immune drink or refreshment.

100 mg-1000 mg Quinine is best per container

1-200 mg of Zinc Vitamin of any variation (50 mg is best for this formula)

1-1000 mg of Vitamin C of any variation (200 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Any Immune Drink with red onions, Baby kale, Cherry Tomatoes and spinach extract of any variation

(Value high Quecertin levels).

Formula 1 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1 Oz.-2 gallon container or any variation for sale

6 Oz.-16 Oz. container is best

Formula 2 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1 Oz.-2 gallon container or any variation for sale

6 Oz.-16 Oz. container is best

Formula 3 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry

Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-1000 mg of Vitamin C of any variation (200 mg is best for this formula)

1 Oz.-2 gallon container or any variation for sale

6 Oz.-16 Oz. container is best

Formula 4 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1-1000 mg of Vitamin C of any variation (200 mg is best for this formula)

1 Oz.-2 gallon container or any variation for sale

6 Oz.-16 Oz. container is best

Formula 5 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1 Oz.-2 gallon container or any variation for sale

6 Oz.-16 Oz. container is best

Formula 6 Red onions, Baby kale, Cherry Tomatoes and spinach extract Immune Drink

1-5000 mg Red onions, Baby kale, Cherry Tomatoes and spinach extract Immune drink in any variation in a drink form. This Includes S Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-1000 mg of Vitamin C of any variation (200 mg is best for this formula)

1 Oz.-2 gallon container or any variation for sale

6 Oz.-16 Oz. container is best

Formula 7 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vita min of any variation (50 mg is best for this formula)

1-200 mcg of vitamin D3 of any variation (25-50 mcg is best for this formula)

1-2000 mg of Calcium of any variation (200-400 mg is best for this formula)

1 Oz.-2 gallon container or any variation for sale

6 Oz.-16 Oz. container is best

Formula 8 Red onions, Baby kale, Cherry Tomatoes and spinach extract Immune Drink

1-5000 mg Red onions, Baby kale, Cherry Tomatoes and spinach extract Immune drink in any variation in a drink form. This Includes Berry, Cherry, or grape Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-1000 mg of Vitamin C of any variation (200 mg is best for this formula)

1 Oz.-2 gallon container or any variation for sale

6 Oz.-16 Oz. container is best

Formula 9 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1 Oz.-2 gallon container or any variation for sale

6 Oz.-16 Oz. container is best

Formula 10 Red onions, Baby kale, Cherry Tomatoes and spinach extract Immune Drink

1-5000 mg Red onions, Baby kale, Cherry Tomatoes and spinach extract Immune drink in any variation in a drink form. This Includes Berry, Cherry, or grape Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1-1000 mg of Vitamin C of any variation (200 mg is best for this formula)

1 Oz.-2 gallon container or any variation for sale

6 Oz.-16 Oz. container is best

Formula 11 Red onions, Baby kale, Cherry Tomatoes and spinach extract Immune Drink

1-5000 mg Red onions, Baby kale, Cherry Tomatoes and spinach extract Immune drink in any variation in a drink form. This Includes Berry, Cherry, or grape Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-200 mg of Zinc Vitamin of any variation (50 mg is best for this formula)

1-200 mcg of vitamin D3 of any variation (25-50 mcg is best for this formula)

1-2000 mg of Calcium of any variation

1-500 mg Sodium Ions such as an energy drink (200 mg Sodium per 16 Oz. is common)

1-300 mg Potassium Ions such as an energy drink (60 mg per 16 per 16 Oz. is common)

1-1000 mg of Vitamin C of any variation (200 mg is best for this formula)

1 Oz.-2 gallon container or any variation for sale

6 Oz.-16 Oz. container is best

Formula 12 Red onions, Baby kale, Cherry Tomatoes and spinach extract Immune Drink

1-5000 mg Red onions, Baby kale, Cherry Tomatoes and spinach extract Immune drink in any variation in a drink form. This Includes Red onions, Baby kale, Cherry Tomatoes and spinach Syrup diluted with fluid or water to make an Immune drink or refreshment. Also includes Red onions, Baby kale, Cherry Tomatoes and spinach extract powder diluted with water or fluid to make an immune drink or refreshment.

100 mg-2000 mg Red onions, Baby kale, Cherry Tomatoes and spinach extract (more specific 1000 mg-1200 mg) is best per container

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container

1-5000 mg Quinine Immune drink in any variation in a drink form. This Includes Quinine Syrup diluted with fluid or water to make an Immune drink or refreshment. Also includes Quinine extract powder diluted with water or fluid to make an immune drink or refreshment.

100 mg-1000 mg Quinine is best per container

1-200 mg of Zinc Vitamin of any variation (50 mg is best for this formula)

1-200 mcg of vitamin D3 of any variation (25-50 mcg is best for this formula)

1-2000 mg of Calcium of any variation (200-400 mg is best for this formula)

1-500 mg Sodium Ions such as an energy drink (200 mg Sodium per 16 Oz. is common)

1-300 mg Potassium Ions such as an energy drink (60 mg per 16 per 16 Oz. is common)

1-1000 mg of Vitamin C of any variation (200 mg is best for this formula)

1 Oz.-2 gallon container or any variation for sale

6 Oz.-16 Oz. container is best

Formula 1 Quercetin and coconut water with or without pulp (with pulp better) Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container (500 mg Quercetin more specific for this formula)

17.8 Oz. Coconut water with or without pulp (with pulp better)

1 Oz.-2 gallon container or any variation for sale

6 Oz.-20 Oz. container is best

Formula 2 Quercetin and coconut water with or without pulp (with pulp better) Immune Drink

1-5000 mg Quercetin Immune drink in any variation in a drink form. This Includes Quercetin Syrup diluted with fluid or water to make an Immune drink or refreshment. Also includes Quercetin extract powder diluted with water or fluid to make an immune drink or refreshment.

100 mg-1000 mg Quercetin is best per container (500 mg Quercetin more specific for this formula)

17.8 Oz. Coconut water with or without pulp (with pulp better)

1-200 mg of Zinc Vitamin of any variation (50 mg is best for this formula)

1 Oz.-2 gallon container or any variation for sale

6 Oz.-20 Oz. container is best

Formula 3 Coconut water with or without pulp (with pulp better) and Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

17.8 Oz. Coconut water with or without pulp (with pulp better)

1 Oz.-2 gallon container or any variation for sale

6 Oz.-20 Oz. container is best

Formula 4 Coconut water with or without pulp (with pulp better)and Berries, Cherries, or Grape extract Immune Drink

1-5000 mg Berries, Cherries, or Grape extract Immune drink in any variation in a drink form. This Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

17.8 Oz. Coconut water with or without pulp (with pulp better)

1-200 mg of Zinc Vitamin of any variation (50 mg is best for this formula)

1 Oz.-2 gallon container or any variation for sale

6 Oz.-20 Oz. container is best

Formula 4 Coconut water with or without pulp (with pulp better)and Berries, Cherries, or Grape extract Immune Drink

1-5000 mg Berries, Cherries, or Grape extract Immune drink in any variation in a drink form. This

Includes Berry, Cherry, or grape Syrup diluted with fluid or water to make an Immune drink or refreshment. Also includes Berry, Cherry, or grape extract powder diluted with water or fluid to make an immune drink or refreshment.

100 mg-2000 mg Berry, Cherry, or Grape extract (more specific 1000 mg-1200 mg) is best per container

17.8 Oz. Coconut water with or without pulp (with pulp better)

1-200 mg of Zinc Vitamin of any variation (50 mg is best for this formula)

1-1000 mg of Vitamin C of any variation (200 mg is best for this formula)

1-200 mcg of vitamin D3 of any variation (25-50 mcg is best for this formula)

1 Oz.-2 gallon container or any variation for sale

6 Oz.-20 Oz. container is best

All Formulas: Can have extra Solvent added to the distilled water and will need sweetener.

½ oz.-1 oz. of Lime and or Lemon Juice was used and added (to 16 oz. drink) as an extra solvent to allow the water to absorb the large volume of vitamins. This helped to make a Homogenous Solution (from Solute to Solvent).

7-40 g. Raw Natural Sugar Cane was used and ground to a fine powder as a sweetener per 16 oz. serving.

¼-1 oz. of Natural Honey can be used as a sweetener. ½ oz. of Natural Honey is best.

7-40 g. Stevia can be used and ground to a fine powder as a diet sweetener per 16 oz. serving.

All drink formulas can be modified from 16 oz. to standard 28 oz. or 32 oz. or any modification to size.

### SUMMARY

My objectives are to patent products with enough volume of mg vitamins to be effective. Also, have perfect recommendation instructions on the container to perfectly allow each consumer to attain quality health. Each product formula with the precise vitamin ingredients for the desired reduction of inflammation and immune support.

### Example: Formula 5 Quercetin, Zinc, D3, Calcium, Sodium, and Potassium Immune Drink

1-5000 mg Quercetin in any variation in a drink form including Quercetin Syrup Diluted with fluid to make an Immune drink or refreshment. 250 mg used in this formula

1-200 mg of Zinc in any variation. 25 mg used in this formula

1-200 mcg of vitamin D3 of any variation. 25 mcg used in this formula

1-2000 mg of Calcium of any variation. 200 mg used in this formula

1-500 mg of Sodium Ions such as in an energy drink (160 mg per 16 oz. is commonly used and is what I use)

1-300 mg of Potassium Ions such as in energy drinks (45 mg per 16 oz. is commonly used and is what I use)

16-32 oz container is best

This Formula is perfect for Immune Support and Reducing Inflammation. There are no other formulas with this much Quercetin, Zinc, D3, Calcium, and Ions like Potassium and Sodium. The idea is to recommend the consumer drink 2-4 16 oz. drinks per day for best results. Two would be sufficient in modern 2021 environment.

This formula is like all my formulas in that they are effective and accurate in the amount. My experience is specific to these formula and there specific ingredients. These formulas will be listed in this patent.

I am a practicing physician who has not only recommended these same vitamin ingredients to 700-1200 patients. I have also monitored there progress along with other health recommendations. My patients received exercise, proper diet, rest and spinal manipulations. My patients and I have achieved amazing results in an inhospitable environment such as we have today. I am perhaps alone in my knowledge and I wish to share these achievements with the world. However, I must first patent my results of my many healthy patients.

1. These vitamin drink formulas, and vitamin combinations are superior for immune support and in reducing inflammation.

2. These vitamin and drink formulas are superior, as in claim 1, working in humans by reducing inflammation due to the inhibition of the TWIK2 Channels (potassium) in the Macrophages (White Blood Cells); this inhibits NIrp3 inflammasome and then in turn inhibits Interleukin Beta signal with in the CNS (central nervous system) that stimulates inflammation.

3. These vitamins and drink formulas work to further reduce inflammation as in claim 1; this happens in the lungs due to the inhibition of TAS2R channels; this inhibits IGE immunoglobulin which reduces allergic inflammation.

4. My vitamin formulas, gummies, and drink formulas are superior further comprising claim 1 in there combinations to what is out there in the markets; this is due to proper Ingredients for cellular changes and higher milligram.

5. These vitamins and drink formulas work to further immune support as in claim 1; this increase in Immune support is the Quinine inhibiting Sars-Covid-2 because of its antiviral activity; this is illustrated in the stomach by inhibiting TMPRSS2 a protease in the colon; sited “Quinine inhibits infection of Human Cell Lines with Sars-Cov-2”; Quercetin is the active bioflavonoid in Quinine and Elderberry.

6. There is an increase in white blood cell and nerve cell proliferation that increases immune support as in claim 1; vitamin D is an Autocrine and Paracrine Proliferation (Making these cells) of B cells, T cells, Macrophages (these first three are White Blood Cells), and Dendrites (nerve cells); these cells can interact with Vitamin D and (Bind) Synthesize 125-Dihydroxy Vitamin D; this causes an increase in white blood cell and nerve cell proliferation that increases immune support.

7. Vitamin D makes Calcium and Bone formation; this increases immune support as in claim 1; vitamin D makes Collagen matrix mineralization of bone; vitamin D is also Skin Synthesized then sent inactive to the Liver; from there D is sent from the Liver to the Kidney as Active vitamin D; bone formation is important in the long bones for the production of white and red blood cells.

8. Vitamin C has antiviral properties that reduces inflammation as in claim 1; vitamin C increases lymphocyte activity, interferon (cell communication) production, modulating cytokines, endothelial function, reduces inflammation, and storing mitochondrial function.

9. Zinc when taken regularly inhibits covid-19 replication.

10. Zinc inhibits viral replication as in claim 9; further comprising Zinc Ionophores such as Quinine, Elderberry, and Quercetin cause an influx of Zinc into human cells; this inhibits viral replication as in claim 9; this is done with plant polyphenols such as flavonoids like Quercetin that act like antioxidants and carry the zinc Ionophore across the cell membrane; Quercetin is a flavonoid in quinine, elderberry, berries, grapes, Kale, Red onions and other fruits and vegetables.

11. Vitamin C Recycles Quercetin Bioflavonoid in the body, Essentially doubling the Quercetin, Quercetin inhibits Polymorases, Proteases Reverse Transcriptase, Suppression DNA Gyrase, and binding Viral Capsid Proteins: this recycling of Quercetin inhibits viral replication.
